(19) United States
(12) Patent Application Publication
(10) Pub. No.: US 2020/0129448 A1
(43) Pub. Date: Apr. 30, 2020

(54) COMPOSITIONS AND METHODS FOR WOUND CLOSURE

(71) Applicant: THROMBO THERAPEUTICS, INC., Walpole, MA (US)

(72) Inventors: Christopher D. BARRETT, Walpole, MA (US); Michael B. YAFFE, West Roxbury, MA (US); Ernest E. MOORE, Denver, CO (US); Hunter B. MOORE, Denver, CO (US); Michael P. CHAPMAN, Denver, CO (US)

(21) Appl. No.: 16/630,384

(22) PCT Filed: Jul. 13, 2018
(86) PCT No.: PCT/US2018/042090

§ 371 Date: Jan. 10, 2020

Related U.S. Application Data
(60) Provisional application No. 62/532,296, filed on Jul. 13, 2017.

(51) Int. Cl. A61K 9/70; A61K 38/36; A61K 38/48; A61K 38/39; A61K 38/00; A61K 38/37; A61K 31/202; A61K 31/351; A61K 45/06; A61K 35/14; A61K 47/34; A61K 35/36; A61K 35/38; A61K 35/12; A61K 31/65; A61K 31/506; A61L 31/10; A61L 29/08

(52) U.S. Cl. CPC A61K 9/7007; A61L 2400/04; A61K 38/4833; A61K 38/39; A61K 38/4813; A61K 38/005; A61K 38/36; A61K 38/37; A61K 31/202; A61K 31/351; A61K 45/06; A61K 35/14; A61K 47/34; A61K 35/36; A61K 35/38; A61K 35/12; A61K 31/65; A61K 31/506; A61L 31/10; A61L 29/085; A61K 38/363

(57) ABSTRACT

The current disclosure provides compositions and methods for wound closure. The disclosure provides an device comprising: a) a material with a surface comprising a modified fibrinogen or a modified fibrin, wherein the modified fibrinogen or modified fibrin is more resistant to fibrinolysis than an unmodified fibrinogen or an unmodified fibrin, respectively; and b) one or more dispersible fibrinolysis inhibitors in contact with the surface comprising a plasmin inhibitor; and c) optionally, a thrombin agent or platelets.

### CROSS-REFERENCE

This application claims priority to U.S. Provisional Application No. U.S. 62/532,296, filed Jul. 13, 2017, which is incorporated herein by reference in their entirety.

### BACKGROUND

Fluid that accumulates in potential spaces made by surgical or other invasive procedures is a strong predictor of risk for surgical site infection. Such procedures can include hernia repairs with or without meshes, device implantation (e.g. venous access ports, pacemakers, cardiac implantable electronic device (CIED), etc.), exploratory laparotomies, and vascular grafts. Surgical site infections can more than double the average length of hospital stays, are responsible for more than 91,000 readmissions to the hospital and nearly 1 million additional hospital days per year at an annual cost of ˜$3.3 billion to manage these infectious complications in the United States alone. Procedures that involve placement of a foreign body (e.g. as hernia repairs with meshes) or implanted materials and devices with direct access to the vascular system (e.g. vascular grafts, venous access ports, pacemakers, and CIED) can be particularly devastating events for patients, the healthcare system and the broader economy when infection occurs due to the significant morbidity and cost increases from recurrent and prolonged hospitalizations, re-operations, chronic wound management, and even death.

Given the association of fluid accumulation (e.g. blood or seroma fluid) in surgically created potential spaces with development of infection, new broadly applicable approaches to reduce fluid accumulation in post-operative wounds are needed, inclusive of the need to ensure blood clot formation (termed “hemostasis”) to prevent blood accumulation which leads to the formation of a hematoma. Such a system would be expected to reduce complications/morbidity (e.g. infections) and reduce costs to patients, healthcare systems, and society.

### SUMMARY

In some embodiments, the disclosure provides an device comprising: a) a material with a surface comprising a modified fibrinogen or a modified fibrin, wherein the modified fibrinogen or modified fibrin is more resistant to fibrinolysis than an unmodified fibrinogen or an unmodified fibrin, respectively; and b) one or more dispersible fibrinolysis inhibitors in contact with the surface comprising a plasmin inhibitor; and c) optionally, a thrombin agent or platelets. In some embodiments, the disclosure provides an implantable or topically applicable device comprising: a) a material with a surface comprising fibrinogen or fibrin attached thereto; and b) one or more dispersable fibrinolysis inhibitors attached to the surface selected from the group consisting of: i) a fibrin and/or fibrinogen modifying agent; and ii) a plasmin inhibitor; c) optionally, a thrombin agent and/or platelets. In some embodiments, the disclosure provides the device wherein the modified fibrinogen or the modified fibrin is more resistant to plasminogen or plasmin cleavage than an unmodified fibrinogen or an unmodified fibrin, respectively. In some embodiments, the disclosure provides the device wherein the device is implantable. In some embodiments, the disclosure provides the device, wherein the device is topically applicable. In some embodiments, the disclosure provides the device wherein the surface further comprises collagen. In some embodiments, the disclosure provides the device wherein the fibrin or fibrinogen modifying agent comprises an enzyme that cleaves the fibrin or fibrinogen. In some embodiments, the disclosure provides the device wherein the fibrin or fibrinogen modifying agent comprises a thrombin-activatable fibrinolysis inhibitor (TAFI). In some embodiments, the disclosure provides the device wherein the fibrin or fibrinogen modifying agent comprises a carboxypeptidase. In some embodiments, the disclosure provides the device wherein the fibrin or fibrinogen modifying agent cleaves a C-terminal amino acid of the fibrin or fibrinogen. In some embodiments, the disclosure provides the device wherein the fibrin or fibrinogen modifying agent cleaves a lysine of the fibrin or fibrinogen. In some embodiments, the disclosure provides the device wherein the plasmin inhibitor comprises a plasmin formation inhibitor. In some embodiments, the disclosure provides the device wherein the plasmin inhibitor comprises a plasmin activity inhibitor. In some embodiments, the disclosure provides the device wherein the plasmin inhibitor comprises a direct or indirect inhibitor of fibrinolysis. In some embodiments, the disclosure provides the device wherein the plasmin inhibitor comprises alpha 2-antiplasmin (A2AP), alpha 2-macroglobulin (A2MG), plasminogen activator inhibitor-1 (PAI-1), plasminogen activator inhibitor-2 (PAI-2)), a lysine analogue, or a combination thereof. In some embodiments, the disclosure provides the device wherein the lysine analogue is aminocaproic acid or tranexamic acid. In some embodiments, the disclosure provides the device wherein the plasmin inhibitor comprises an inhibitor of uPA. In some embodiments, the disclosure provides the device wherein the plasmin inhibitor comprises an inhibitor of tPA. In some embodiments, the disclosure provides the device wherein the plasmin inhibitor comprises an inhibitor of streptokinase. In some embodiments, the disclosure provides the device wherein the plasmin inhibitor comprises an inhibitor of plasmin. In some embodiments, the disclosure provides the device wherein the plasmin inhibitor or a plasmin activity inhibitor agent is a small molecule. In some embodiments, the disclosure provides the device wherein the plasmin inhibitor or a plasmin activity inhibitor agent is an antibody. In some embodiments, the disclosure provides the device wherein the thrombin agent comprises thrombin, prothrombin, or derivatives thereof. In some embodiments, the disclosure provides the device wherein the thrombin agent comprises an inhibitor of TFPI, an inhibitor of Protein S, or an inhibitor of activated Protein C, such as a natural or recombinant protein or other pharmacologic agent. In some embodiments, the disclosure provides the device wherein the thrombin agent comprises alpha-thrombin. In some embodiments, the disclosure provides the device wherein the thrombin agent is a natural or recombinant protein that is resistant to inactivation by anti-thrombin, oral direct thrombin, such as dabigatran or efegatran, or injected thrombin inhibitors, such as argatroban, bivalirudin or hirudin. In some embodiments, the disclosure provides the device wherein the device further comprises fibrin or fibrinogen linking agent. In some embodiments, the disclosure provides the device wherein the fibrin or fibrinogen linking agent links the fibrin or fibringogen to platelets. In some embodiments, the disclosure provides the device wherein the fibrin or fibrinogen linking agent comprises a Von Willebrand Factor (vWF). In some embodiments, the disclosure provides the device wherein the device further comprises a platelet activating agent. In some embodiments, the disclosure provides the device wherein the platelet activating agent comprises arachidonic acid or thromboxane. In some embodiments, the disclosure provides the device wherein the platelet activating agent comprises a coagulation factor. In some embodiments, the disclosure provides the device wherein the coagulation factor comprises factor X, factor Xa, factor V, factor Va, factor XII, factor XIIa, factor XI, factor XIa, factor IX, factor IXa, factor VIIIa, Tissue Factor, an inhibitor of Activated Protein C, an inhibitor of Protein S, an inhibitor of an anticoagulant, or an inhibitor of injected factor Xa. In some embodiments, the disclosure provides the device wherein the platelet activating agent is a recombinant protein. In some embodiments, the disclosure provides the device wherein the factor V is a R506Q variant, a R534Q variant, resistant to cleavage or inactivation by Activated Protein C, a variant with increased activity compared to natural factor V, or a constitutively active variant. In some embodiments, the disclosure provides the device wherein the factor X or factor Xa is resistant to inactivation, a variant with increased activity compared to natural Factor X or Xa, or a constitutively active variant. In some embodiments, the disclosure provides the device wherein the factor X or factor Xa is resistant to inactivation in the presence of a heparin agent, such as unfractionated heparin, enoxaparin, dalteparin, fondaparinux, or tinzaparin. In some embodiments, the disclosure provides the device wherein the factor X or factor Xa is resistant to inactivation by an oral Factor Xa inhibitor, such as apixaban, rivaroxaban, betrixaban, darexaban, edoxaban, otamixaban, letaxaban, or eribaxaban. In some embodiments, the disclosure provides the device wherein the factor VII or factor VIIa is resistant to inactivation, a variant with increased activity compared to natural VII or factor VIIa, a constitutively active variant or a variant with increased protease activity with or without association with Tissue Factor. In some embodiments, the disclosure provides the device wherein the Tissue Factor is resistant to inactivation, a variant with increased activity compared to natural Tissue Factor, a constitutively active variant or a variant with increased protease activity. In some embodiments, the disclosure provides the device wherein the factor VIII or factor VIIIa is a constitutively active variant, functions as a cofactor for Factor IXa independent of cleavage events, or a variant more resistant to inactivation, such as inactivation by Activated Protein C. In some embodiments, the disclosure provides the device wherein the factor IX or factor IXa is a constitutively active variant, variant more resistant to inactivation, or a variant with increased protease activity with or without association with Factor VIIIa or similar derivatives. In some embodiments, the disclosure provides the device wherein the inhibitor of Activated Protein C is a recombinant protein or other pharmacologic agent that inhibits activity of the Activated Protein C. In some embodiments, the disclosure provides the device wherein the inhibitor of Protein S is antibody, nanoparticle, or other pharmacologic agents that blocks, inhibits, or sequesters Protein S. In some embodiments, the disclosure provides the device wherein the inhibitor of the anticoagulant is an antibody, nanoparticle, molecular decoy, decoy receptor, or other pharmacologic sequestration/binding agent that binds, blocks, inhibits, or sequesters oral anticoagulants or oral Factor Xa inhibitor, or a protein or molecule that mimics Factor II or Factor IIa to bind/sequester an oral direct thrombin inhibitor or an injected thrombin inhibitor. In some embodiments, the disclosure provides the device wherein the inhibitor of the anticoagulant is apixaban, rivaroxaban, betrixaban, darexaban, edoxaban, otamixaban, letaxaban, eribaxaban, Andexanet alfa, rivaroxaban, apixaban, dabigatran, efegatran, argatroban, bivalirudin, hirudin, a protein or molecule that mimics anti-thrombin to bind/sequester/inactivate a heparin agent such as unfractionated heparin or enoxiparin, or a monoclonal antibody such as idarucizumab that binds dabigatran. In some embodiments, the disclosure provides the device wherein the inhibitor of the anticoagulant is ciraparantag (“PER977,” IUPAC name N¹,N^{1′}-[Piperazine-1,4-diylbis(propane-1,3-diyl)]bis-L-argininamide) or a compound that reverses anticoagulant molecules, such as rivaroxaban, apixaban, dabigatran, unfractionated heparin, and low molecular weight heparins. In some embodiments, the disclosure provides the device wherein the inhibitor of the anticoagulant is an antibody, such as a divalent antibody, that targets a basement membrane protein such as TF or COL4, sequesters an anticoagulant, such as an anticoagulant delivered deliver topically or by IV. In some embodiments, the disclosure provides the device wherein the inhibitor of the anticoagulant is an antibody, such as a divalent antibody or modified antibody that targets an organ's endothelium and contains an anticoagulant such that anticoagulation is localized to an organ of interest. In some embodiments, the disclosure provides the device wherein the inhibitor of injected factor Xa is a protein, antibody, or other pharmacologic molecule that binds, blocks, inhibits, or sequesters injected Factor Xa inhibitors, such as enoxaparin, dalteparin, fondaparinux, tinzaparin, protamine, or an anti-thrombin-like protein. In some embodiments, the disclosure provides the device wherein the coagulation factor comprises a lipid, such as a negatively charged phospholipid. In some embodiments, the disclosure provides the device wherein the coagulation factor comprises aprotinin. In some embodiments, the disclosure provides the device wherein the platelet activating agent is a natural protein. In some embodiments, the disclosure provides the device wherein the device further comprises a fibrin cross-linking agent. In some embodiments, the disclosure provides the device wherein the fibrin cross-linking agent comprises Factor XIII In some embodiments, the disclosure provides the device wherein the device further comprises a pain control agent or anesthetic agent (e.g. delayed-release liposomal bupivacaine, bupivacaine, lidocaine, xylocaine, etc.). In some embodiments, the disclosure provides the device wherein the device further comprises an antibiotic, such as a bacterial cell wall synthesis inhibitor such as vancomycin or penicillin or derivatives thereof, aminoglycosides such as gentamicin, fluoroquinolones such as ciprofloxacin, macrolides such as erythromycin, other ribosomal inhibitors such as tetracycline. In some embodiments, the disclosure provides the device wherein the device further comprises a patient blood product. In some embodiments, the disclosure provides the device wherein the fibrinogen or fibrin is attached to the surface covalently. In some embodiments, the disclosure provides the device wherein the fibrinogen or fibrin is attached to the surface noncovalently. In some embodiments, the disclosure provides the device wherein the one or more dispersible fibrinolysis inhibitors agents are recombinant proteins, modified recombinant proteins, native proteins, and/or modified native proteins, with or without pharmacologic agent(s). In some embodiments, the disclosure provides the device wherein the material is a film, sheet, patch, device, vascular graft, mesh, mesh-like material, or a scaffold-like material. In some embodiments, the disclosure provides the device wherein the material is biologic, synthetic, biosynthetic or a combination of biologic and/or synthetic and/or biosynthetic origin. In some embodiments, the disclosure provides the device wherein the material is polyester, polypropylene, polytetrafluoroethylene, polyglactin 910, or poliglecaprone 25. In some embodiments, the disclosure provides the device wherein the material is an implanted venous access catheter or port, pacemaker, or CIED. In some embodiments, the disclosure provides the device wherein the material is porous. In some embodiments, the disclosure provides the device wherein the material is non-porous. In some embodiments, the disclosure provides the device wherein the material is absorbable. In some embodiments, the disclosure provides the device wherein the material is non-absorbable. In some embodiments, the disclosure provides the device wherein the material is made from dermis, pericardium, intestinal wall, collagen, fibrinogen, fibrinogen derivative, or fibrin. In some embodiments, the disclosure provides the device wherein the material is biosynthetic such as a mixture of polyglycolic acid and trimethylene carbonate or a mixturesof polyglycolic acid, polylactic acid, and trimethylene carbonate, or another polymer mixture. In some embodiments, the disclosure provides the device wherein the material comprises minocycline, rifampin or both. In some embodiments, the disclosure provides the device wherein the inocycline, rifampin or both is releasable from the material. In some embodiments, the disclosure provides the device wherein the implantable device is a material, device, mesh, mesh-like material, or scaffold. In some embodiments, the disclosure provides the device wherein the device is a mesh. In some embodiments, the disclosure provides the device wherein the device is elastic. In some embodiments, the disclosure provides the device wherein the device is radially elastic. In some embodiments, the disclosure provides the device wherein the device is an orthopedic implant. In some embodiments, the disclosure provides the device wherein the material has a substantially uniform Young's modulus constant throughout. In some embodiments, the disclosure provides the device wherein a thickness of the material is varied, wherein an overall radial spring constant decreases with distance from the center. In some embodiments, the disclosure provides the device wherein the material comprises woven fibers, wherein a geometric stiffness of the fibers is varied along a radius of the material by varying a cross-sectional shape of the fibers without changing a weave of the fibers. In some embodiments, the disclosure provides the device wherein the material comprises an unwoven mesh of fibers, wherein a deposition isometry of the fibers is varied such that the fibers are radially aligned near a center of the material and/or increase in random alignment toward an edge of the material edge. In some embodiments, the disclosure provides the device wherein the material comprises a mesh made of fibers made of 2 or more substances or 2 or more cross-sectional profiles. In some embodiments, the disclosure provides the device wherein a blend ratio of the mesh made of fibers varies from a center of the surface to an edge of the surface. In some embodiments, the disclosure provides the device wherein the material comprises a mesh and wherein an elasticity of the mesh across a contour is varied. In some embodiments, the disclosure provides the device wherein the material comprises a mesh made of fibers that is microscopically textured such that interwoven fibers are free to, move, slip, or stretch amongst themselves. In some embodiments, the disclosure provides the device wherein the material comprises a mesh with two sides that are free to, move, slip, or stretch with respect to each other. In some embodiments, the disclosure provides the device wherein the fibrin or fibrinogen attached to the surface is natural, recombinant, or post-translationally modified. In some embodiments, the disclosure provides the device wherein the fibrin or fibrinogen is resistant to fibrinolysis/clot breakdown.

In some embodiments, the disclosure provides a method of sealing a wound comprising inserting the device into a space of the wound. In some embodiments, the disclosure provides a method wherein the method further comprises closing the wound. In some embodiments, the disclosure provides a method wherein the wound is closed by a medical device or tool comprising surgical graspers, hooks, forceps, robot, sutures, staples, tacks, synthetic, biologic glue, or strips of tape. In some embodiments, the disclosure provides a method wherein the wound is closed by firm pressure. In some embodiments, the disclosure provides a method wherein the method further comprises dispersing an agent. In some embodiments, the disclosure provides a method wherein the agent comprises fibrinolysis inhibitors, fibrin or fibrinogen modifying agents, plasmin inhibitors, inhibitor of anticoagulants, platelet activating agents, or any combinations thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates exemplary prosthetics disclosed in the current invention.

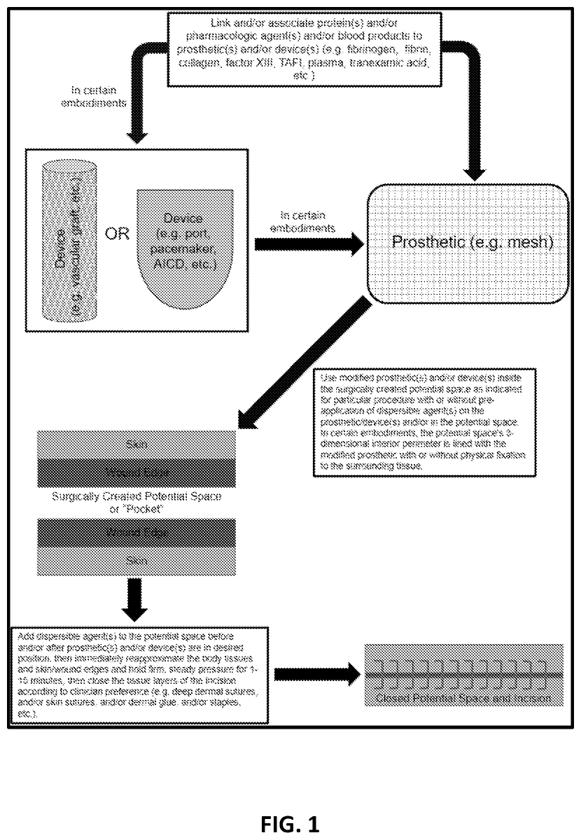

FIG. 2 illustrates exemplary prosthetics disclosed in the current invention.

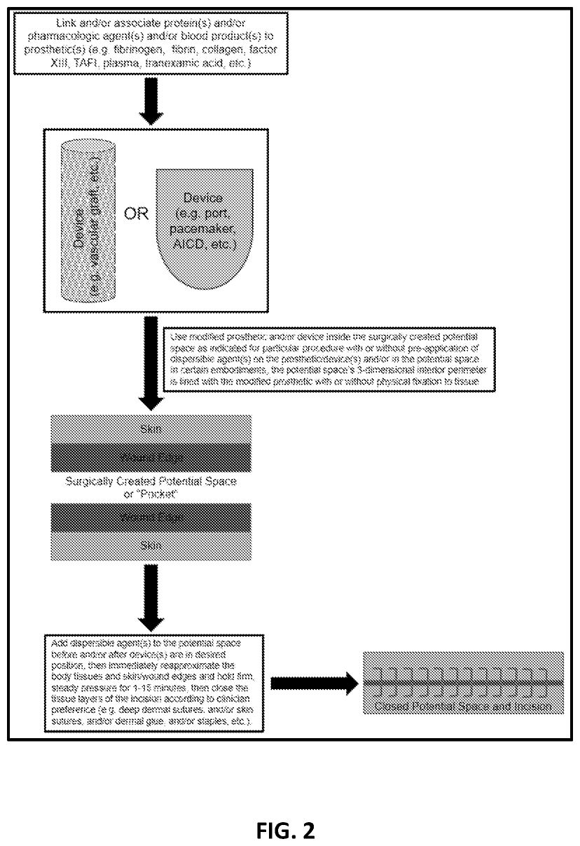

FIG. 3 illustrates a simplified model of neutrophil elastase in coagulation and fibrinolysis.

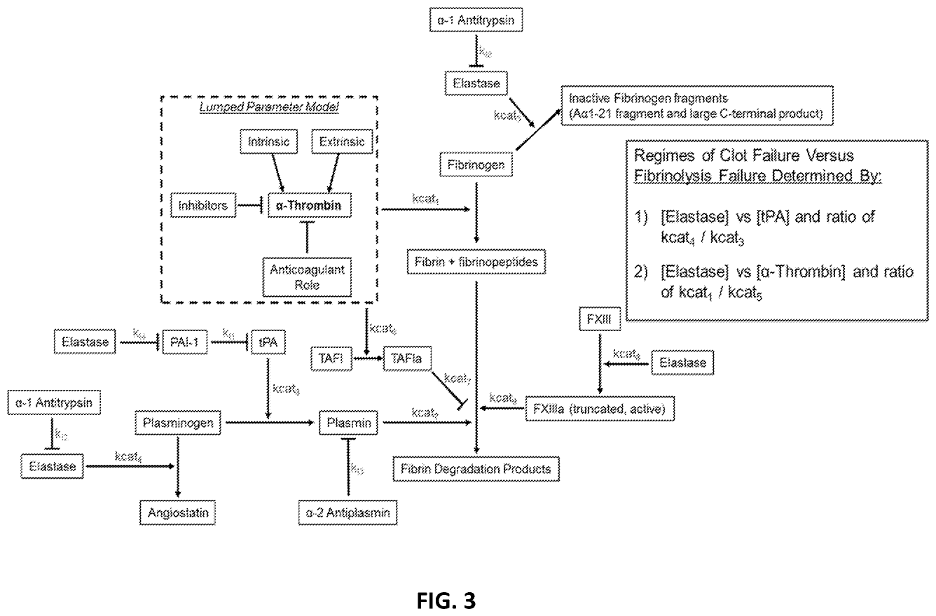

### DETAILED DESCRIPTION

It is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

The term “about” or “approximately or approx.” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. The details of one or more particular embodiments are set forth in the description below.

### Definitions

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

A “prosthesis” (plural: prostheses) or “prosthetic” can refer to an artificial device, either external or implanted, that replaces or supplements a missing or a defective body part, which may be lost or damaged through trauma, disease, or congenital conditions. It can be a material, a device, a mesh or mesh-like material, or a scaffold.

A “kit” as used herein, can define a package or an assembly including one or more of the components of the invention, and/or other components associated with the invention, for example, as previously described. A kit of the invention may, in some cases, include instructions in any form that are provided in connection with the components of the invention in such a manner that one of ordinary skill in the art would recognize that the instructions are to be associated with the components of the invention.

A “potential space” or “surgically created potential space” used herein can be used interchangeably, and can refer to a space that can occur between two adjacent structures that are normally pressed together. A surgically created potential space can be soft tissue space or cavity.

Cardiovascular implantable electronic devices (CIEDs) can include permanent pacemakers (PPMs), implantable cardioverter-defibrillators (ICDs) and cardiac resynchronization therapy (CRT-D [with defibrillator] and CRT-P [without defibrillator]) in improving quality of life and survival. CIED can refer to automatic implantable cardioverter-defibrillator (AICD).

A “protein” used herein can refer to a recombinant protein, modified recombinant protein, native protein, or modified native protein. A protein can be any derivatives of a protein. A protein can be full-length or a fragment of the full-length protein.

An “agent” or “modifying agent” used herein can refer to an agent that can directly or indirectly promote hemostasis. The agent can be covalently or non-covalently linked to, or impregnated in, or otherwise associated with a prosthetic. The agent can also be a dispersible agent.

As used herein, “recombinant” can refer to the alteration of genetic material by human intervention. Typically, recombinant can refer to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” nucleic acid can be described with reference to how it differs from a naturally occurring counterpart (the “wild-type”). Recombinant protein can refer to a protein expressed by recombinant DNA technology.

The term “molecular decoy” or “molecular decoy receptor” can be used interchangeably, and can refer to molecules which bind to and interfere with a biological function of a target. The molecular decoys can compete with its natural counterpart and bind, block, inhibit, or sequester a target.

### Overview

Novel approaches have been developed in recent years to reduce fluid accumulation and infection in surgically created potential spaces. One of these novel approaches is to apply an external vacuum device to the wound to suction the accumulating fluid out of the wound through the closed incision. However, this approach does not provide any mechanism to augment hemostasis, and has been reported to fail to improve the outcomes. Others have attempted to use “fibrin sealant” or “fibrin glue” in surgical procedures. For example, in mesh implantation, “fibrin sealant” or “fibrin glue” can be used as a method to hold the mesh in place and reduce fluid accumulation in the wound. While these approaches such as fibrin sealant or fibrin blue used one particular aspect of the coagulation system (i.e. fibrin) to reduce complications, they failed to take advantage of the multiple fibrin modifying and other blood clot modifying components and nuances of the coagulation system to form a tight hemostatic seal that functionally obliterates any potential space in the surgical wound. Another approach focused on utilizing a combination of an implantable material (e.g. biologic or biosynthetic materials including mesh) with or without protein modifications on its surface in combination with application of other protein mixtures including coagulation proteins, a fixation device (e.g. stapler, sutures, etc.), and a required application of energy (e.g. a surgical radiofrequency device, laser energy, ultraviolet energy, etc.) to seal body tissues together in such a way that they would not leak. However, this approach has several major limitations that prevent its clinical utility for the problem of fluid accumulation in surgical potential spaces, especially surgical potential spaces that contain a prosthetic or device implant, the most notable of which is the requirement in this method for the application of an energy source to incorporate the components that make the “seal” with the body tissues.

To address this major clinical problem, the current disclosure provides a novel approach that can rapidly and durably reduce the size of a surgically created potential space within the body in a manner that simultaneously provides hemostasis, thereby reducing fluid accumulation and bleeding in surgical wounds. This approach is termed Surgical Technologies to Obliterate Potential Surgical Space, and hereafter referred as “STOPS”. In some embodiments, the current disclosure provides a surface-modified prosthetic wherein the modifications can contain agents that generate fibrin clots to promote hemostasis. In some embodiments, the current disclosure provides a surface-modified prosthetic wherein the modifications can contain agents that generate modified fibrin clots that are resistant to breakdown. In some embodiments, the current disclosure provides a surface-modified prosthetic wherein the modifications contain one or more inhibitors of fibrin clot breakdown. In some embodiments, the current disclosure provides a surface-modified prosthetic wherein the modifications can contain both agents that promote hemostasis and one or more inhibitors of fibrin clot breakdown. In some embodiments, the current disclosure provides a surface-modified prosthetic wherein the modifications further comprise collagen. In some embodiments, the current disclosure provides a surface-modified prosthetic wherein the modifications further comprise a dispersible agent. The prosthetic provided herein can be an implantable or topically applicable device or material. The prosthetic can be either modified directly on its surface or can be placed within another prosthetic that has the above mentioned surface modifications. In some embodiments, the prosthetic can be both modified on its surface and placed within another surface-modified prosthetic.

The current disclosure also provides a kit containing the disclosed prosthetic. The current disclosure further provides methods of using the disclosed prosthetic to promote wound closure. The hemostatic fibrin clots, which form upon performance of STOPS, can eliminate the surgical potential space by forming durable and lysis-resistant fibrin clots that can hold the surrounding wound tissue (i.e. the tissue that makes up the 3-dimensional perimeter of the surgical potential space) in apposition with the surface-modified prosthetic.

Hemostasis and Coagulation

The current disclosure provides compositions and methods of using the compositions to promote wound closure wherein the composition can comprise a surface-modified prosthetic to promote hemostasis or coagulation. The disclosed prosthetic can be modified with an agent that either directly or indirectly promote coagulation.

Hemostasis, the arrest of bleeding from an injured blood vessel, can be achieved through the combined activity of vascular, platelet, and plasma factors. Coagulation (also known as clotting) is the process by which blood changes from a liquid to a gel, forming a blood clot, which can potentially result in hemostasis. Regulatory mechanisms can counterbalance the tendency of clots to form. Hemostatic abnormalities can lead to excessive bleeding or thrombosis.

Vascular factors can reduce blood loss due to trauma through local vasoconstriction (an immediate reaction to injury) and compression of injured vessels by extravasation of blood into surrounding tissues. Vessel wall injury can trigger the attachment and activation of platelets and production of fibrin; platelets and fibrin can combine to form a clot.

Various mechanisms, including endothelial cell nitric oxide and prostacyclin, can promote blood fluidity by preventing platelet stasis and dilating intact blood vessels. These mediators may no longer be produced when the vascular endothelium is disrupted. Under these conditions, platelets can adhere to the damaged intima and form aggregates. Initial platelet adhesion is to von Willebrand factor (VWF), previously secreted by endothelial cells into the subendothelium. VWF can bind to receptors on the platelet surface membrane (glycoprotein Ib/IX). Platelets anchored to the vessel wall undergo activation. During activation, platelets can release mediators from storage granules, including adenosine diphosphate (ADP). Other biochemical changes resulting from activation can include hydrolysis of membrane phospholipids, inhibition of adenylate cyclase, mobilization of intracellular calcium, and phosphorylation of intracellular proteins. Arachidonic acid is converted to thromboxane A2; this reaction requires cyclooxygenase and can be inhibited irreversibly by aspirin and reversibly by many NSAIDs. ADP, thromboxane A2, and other mediators induce activation and aggregation of additional platelets on the injured endothelium. Another receptor is assembled on the platelet surface membrane from glycoproteins IIb and Ma. Fibrinogen can bind to the glycoprotein IIb/IIIa complexes of adjacent platelets, connecting them into aggregates. Platelets can provide surfaces for the assembly and activation of coagulation complexes and the generation of thrombin. Thrombin can convert fibrinogen to fibrin. Fibrin strands can bind aggregated platelets to help secure the platelet-fibrin hemostatic plug.

Plasma coagulation factors can interact to produce thrombin, which converts fibrinogen to fibrin. By radiating from and anchoring the hemostatic plug, fibrin can strengthen the clot. In the intrinsic pathway, factor XII, high molecular weight kininogen, prekallikrein, and activated factor XI (factor XIa) can interact to produce factor IXa from factor IX. Factor IXa can then combine with factor VIIIa and procoagulant phospholipid which can be present on the surface of activated platelets and tissue cells to form a complex that can activate factor X. In the extrinsic pathway, factor VIIa and tissue factor (TF) can directly activate factor X and can also activate factor IX.

Activation of the intrinsic or extrinsic pathway can activate the common pathway, resulting in formation of the fibrin clot. Three steps can be involved in common pathway activation: (1) A prothrombin activator is produced on the surface of activated platelets and tissue cells. The activator is a complex of an enzyme, factor Xa, and 2 cofactors, factor Va and procoagulant phospholipid. (2) The prothrombin activator cleaves prothrombin into thrombin and another fragment. (3) Thrombin induces the generation of fibrin polymers from fibrinogen. Thrombin can also activate factor XIII, an enzyme that catalyzes formation of stronger bonds between adjacent fibrin monomers, as well as activating factor VIII and factor XI.

Calcium ions are needed in most thrombin-generating reactions. Calcium-chelating agents, for example, citrate and ethylenediaminetetraacetic acid, can be used in vitro as anticoagulants. Vitamin K-dependent clotting factors (factors II, VII, IX, and X) cannot bind normally to phospholipid surfaces through calcium bridges and function in blood coagulation when the factors are synthesized in the absence of vitamin K.

Although the coagulation pathways can be helpful in understanding mechanisms and laboratory evaluation of coagulation disorders, in vivo coagulation can be predominantly via the extrinsic pathway. People with hereditary deficiencies of factor XII, high molecular weight kininogen, or prekallikrein may have no bleeding abnormality. People with hereditary factor XI deficiency may have a mild to moderate bleeding disorder. In vivo, factor XI (an intrinsic pathway factor) can be activated when a small amount of thrombin is generated. Factor IX can be activated both by factor XIa and factor VIIa/tissue factor complexes.

In vivo, initiation of the extrinsic pathway can occur when injury to blood vessels brings blood into contact with tissue factor on membranes of cells within and around the vessel walls. This contact with tissue factor can generate factor VIIa/tissue factor complexes that activate factor X and factor IX. Factor IXa, combined with its cofactor, factor VIIIa, on phospholipid membrane surfaces can generate additional factor Xa. Factor X activation by both factor VIIa/tissue factor and factor IXa/VIIIa complexes may be required for normal hemostasis. This requirement for factors VIII and IX can explain why hemophilia type A (deficiency of factor VIII) or type B (deficiency of factor IX) results in bleeding despite an intact extrinsic coagulation pathway initiated by factor VIIa/tissue factor complexes.

Several inhibitory mechanisms can prevent activated coagulation reactions from amplifying uncontrollably, causing extensive local thrombosis or disseminated intravascular coagulation. These mechanisms can include inactivation of procoagulant enzymes, fibrinolysis, and ⋅Hepatic clearance of activated clotting factors.

Plasma protease inhibitors (e.g. antithrombin, tissue factor pathway inhibitor, α2-macroglobulin, heparincofactor II) can inactivate coagulation enzymes. Antithrombin inhibits thrombin, factor Xa, factor XIa, and factor IXa. Two vitamin K-dependent proteins, protein C and free protein S, can form a complex that can inactivate factors VIIIa and Va by proteolysis. Thrombin, when bound to a receptor on endothelial cells (thrombomodulin), can activate protein C. Activated protein C, in combination with free protein S and phospholipid cofactors, can proteolyze and inactivate factors VIIIa and Va. In addition to intrinsic inactivators, there are a number of anticoagulant drugs that potentiate the inactivation of coagulation factors. For example, heparin can enhance antithrombin activity. Warfarin is a vitamin K antagonist, and can inhibit regeneration of the active form of vitamin K and, therefore, inhibit generation of functional forms of the vitamin K-dependent clotting factors II, VII, IX and X. Unfractionated heparin (UFH) and low molecular weight heparins (LMWH) can enhance activity of antithrombin and inactivate factors IIa (thrombin) and Xa. LMWHs can include enoxaparin, dalteparin, and tinzaparin. Fondaparinux is a small, synthetic molecule, containing the essential pentasaccharide portion of the heparin structure that can enhance antithrombin inactivation of factor Xa (but not IIa). Parenteral direct thrombin inhibitors can include argatroban and lepirudin. The newer oral anticoagulants can include oral direct thrombin inhibitors (dabigatran) and oral direct factor Xa inhibitors (apixaban, rivaroxaban, edoxaban).

Fibrin deposition and lysis are balanced in vivo to maintain temporarily, and subsequently remove, the hemostatic seal during repair of an injured vessel wall. The fibrinolytic system can dissolve fibrin by means of plasmin, a proteolytic enzyme. Fibrinolysis can be activated by plasminogen activators released from vascular endothelial cells. Plasminogen activators and plasminogen (from plasma) can bind to fibrin, and plasminogen activators can cleave plasminogen into plasmin. Plasmin can then proteolyze fibrin into soluble fibrin degradation products that can be swept away in the circulation.

Several exemplary plasminogen activators are provided herein. For example, tissue plasminogen activator (tPA), from endothelial cells, may be a poor activator when free in solution but can be an efficient activator when bound to fibrin in proximity to plasminogen. Urokinase can exist in single-chain and double-chain forms with different functional properties. Single-chain urokinase may not activate free plasminogen but, like tPA, can readily activate plasminogen bound to fibrin. A trace concentration of plasmin can cleave single-chain to double-chain urokinase, which can activate plasminogen in solution as well as plasminogen bound to fibrin. Epithelial cells that line excretory passages (e.g., renal tubules, mammary ducts) can secrete urokinase, which can be the physiologic activator of fibrinolysis in these channels. Streptokinase, a bacterial product not normally found in the human body, is another exemplary potent plasminogen activator.

Fibrinolysis can be regulated by plasminogen activator inhibitors (PAIS) and plasmin inhibitors that slow fibrinolysis. PAI-1 can inactivate tPA and urokinase and can be released from vascular endothelial cells and activated platelets. The primary plasmin inhibitor can be alpha2-antiplasmin, which can inactivate any free plasmin escaping from clots. Some alpha2-antiplasmin can be cross-linked to fibrin polymers by the action of factor XIIIa during clotting. This cross-linking may prevent excessive plasmin activity within clots. tPA and urokinase are rapidly cleared by the liver, which is another mechanism of preventing excessive fibrinolysis.

Modifications and Modifying Agents

The current disclosure provides a prosthetic that is modified with an agent to promote hemostasis at a wound site. There can be two types of agents. One can be any agents that can positively affect coagulation or promote clotting, such as fibrin, fibrinogen, and thrombin. The other can be any agents that can negatively regulate the fibrinolysis, such as a plasmin formation inhibiting agent. The agents that can positively affect coagulation can further comprise inhibitors of anticoagulants. The agents that can negatively regulate the fibrinolysis can further comprise fibrin and/or fibrinogen modifying agents. For example, the fibrin and/or fibrinogen modifying agents can modify fibrin clots such that the clots are resistant to fibrinolysis. In some embodiments, the disclosed prosthetic can comprise an agent that can render fibrin clot resistant to fibrinolysis. In some other embodiments, the disclosed prosthetic can comprise an agent that can directly or indirectly inhibit fibrinolysis to prevent break down of the fibrin clot. In some other embodiments, the disclosed prosthetic can comprise an agent that can cleave fibrinogen to fibrin and/or otherwise cause fibrin or fibrin-like proteins to polymerize or link together. The link can be covalent or non-covalent which can lead to the formation of fibrin clots that can hold the 3-dimensional perimeter of the potential space in apposition with the prosthetic to eliminate the potential space or significantly reduce the size of the potential space. Further, in some embodiments, the disclosed prosthetic can comprise an agent to help link platelets to fibrin and/or fibrinogen. In certain embodiments, the disclosed prosthetic can comprise an agent to help attract and activate additional platelets to the surgically created potential space to provide additional thrombin generation.

The disclosed prosthetic can comprise an agent that promote clotting. In some embodiments, the disclosed prosthetic can be modified on its surface an agent that can promote clotting. The disclosed prosthetic can also comprise an agent that inhibit fibrinolysis. In some embodiments, the disclosed prosthetic can be modified on its surface an agent that inhibit fibrinolysis. In certain embodiments, the disclosed prosthetic can comprise both an agent that promote clotting and an agent that inhibit fibrinolysis. The agent can be natural, recombinant, modified, or a combination thereof.

Agents that can promote clotting can be called hemostatic agents. These agents can included, but not limited to, aminocaproic acid, tranexamic acid, aprotinin, desmopressin, topical hemostatic agents, tissue adhesives, and vitamin K (Phytonadione). The topical hemostatic agent can include pads, powers, pastes, sponges, solutions, meshes and special dressings that may be used before and during surgery to control blood loss from open wounds by promoting the clotting of whole blood, or plasma. In many types of surgery, several hemostatic agents may be combined for better hemostasis. Commercially available thrombin can be of bovine or human origin. Tissue adhesives can be products used to decrease blood loss. Fibrin glue is a human-derived tissue adhesive that may be used for hemostasis and sealing of tissues. This biological glue can be manufactured from clotting factors taken from donor plasma (fibrinogen and thrombin) or made intraoperatively out of fibrinogen coming from the patient's own blood. Tissue adhesives may be used topically or to seal wound surfaces to reduce postoperative bleeding, decrease or eliminate the need for sutures, as well as in treating thermal injuries. Recombinant antihemophilic (clotting) factors are biosynthetic forms of endogenous (naturally occurring) human blood coagulation factors. They are prepared using recombinant DNA technology (genetically engineered) and produce the same biological effects as the corresponding plasma-derived clotting products. Exemplary recombinant antihemophilic factors include recombinant factor VIIa, recombinant factor VIII, recombinant factor IX.

In some embodiments, the disclosed prosthetic comprises fibrinogen. The fibrinogen can include natural, recombinant, and post-translationally processed forms of fibrinogen that are resistant to fibrinolysis/clot breakdown. For example, various forms of fibrinogen can include “gamma prime” fibrinogen, fibrinogen mutated to be missing its C-terminal lysine residues, or fibrinogen processed via carboxypeptidase digestion (e.g. with thrombin-activatable fibrinolysis inhibitor, or porcine pancreas carboxypeptidase B) to be missing its C-terminal lysine residues.

In some embodiment, the disclosed prosthetic comprises natural and/or recombinant Factor V Leiden (e.g. R506Q and R534Q variants) and other natural variants or recombinant Factor V derivatives resistant to cleavage/inactivation by Activated Protein C. In some embodiments, the recombinant Factor V can be hyperactive or constitutively active variants.

In some embodiments, the disclosed prosthetic comprises natural and/or recombinant Factor X or its derivatives. In some embodiments, the disclosed prosthetic comprises national and/or recombinant Factor Xa or its derivatives. These factor or factor derivatives can include constitutively active variants (with or without the need for Factor Va co-association) and variants more resistant to inactivation. For example, these variants can be resistant to inactivation by anti-thrombin agents such as heparin agents, or resistant to inactivation by oral Factor Xa inhibitors. Exemplary heparin agents include, but are not limited to, unfractionated heparin, enoxaparin, dalteparin, fondaparinux, and tinzaparin. Exemplary oral Factor Xa inhibitors include, but are not limited to, apixaban, rivaroxaban, betrixaban, darexaban, edoxaban, otamixaban, letaxaban, and eribaxaban.

In some embodiments, the disclosed prosthetic comprises natural and/or recombinant prothrombin or alpha-thrombin or thrombin-like proteins (e.g. proteins from venom of snakes of the Bothrops genus like B. moojeni, B. atrox) that are resistant to inactivation by anti-thrombin agents. These anti-thrombin agents can include oral direct thrombin inhibitors such as dabigatran and efegatran, or injected thrombin inhibitors such as argatroban, bivalirudin and hirudin.

In some embodiments, the disclosed prosthetic comprises natural and/or recombinant Factor VII or Factor VIIa proteins or their derivatives, including constitutively active variants, variants more resistant to inactivation, and variants with increased protease activity with or without association with Tissue Factor.

In some embodiments, the disclosed prosthetic comprises natural and/or recombinant Tissue Factor or its derivatives.

In some embodiments, the disclosed prosthetic comprises natural and/or recombinant Factor VIII or Factor VIIIa proteins or their derivatives, including constitutively active variants that function as cofactors for Factor IXa independent of cleavage events, and variants more resistant to inactivation by Activated Protein C or other inactivating events.

In some embodiments, the disclosed prosthetic comprises natural and/or recombinant Factor IX or Factor IXa proteins or their derivatives, including constitutively active variants and variants more resistant to inactivation, and including variants with increased protease activity with or without association with Factor VIIIa or similar derivatives.

In some embodiments, the disclosed prosthetic comprises natural or recombinant proteins or other pharmacologic agents that inhibit that activity of Activated Protein C.

In some embodiments, the disclosed prosthetic comprises antibodies, nanoparticles, or other agents that can block, inhibit, or sequester Protein S.

In some embodiments, the disclosed prosthetic comprises antibodies, nanoparticles, molecular “decoys” or “decoy receptors,” or other binding agents that can bind, block, inhibit, or sequester oral anticoagulants such as oral direct thrombin inhibitors (e.g. dabigatran, efegatran, inogatran, melagatran, ximelagatran) and oral Factor Xa inhibitors (e.g. apixaban, rivaroxaban, betrixaban, darexaban, edoxaban, otamixaban, letaxaban, and eribaxaban). For example, Andexanet alfa can mimic Factor Xa to sequester oral Factor Xa inhibitors such as rivaroxaban and apixaban. For another example, a protein/molecule can be used to mimic Factor II (prothrombin) or Factor IIa (“thrombin”) to sequester oral direct thrombin inhibitors (e.g. dabigatran, efegatran, etc.) or injected thrombin inhibitors (e.g. argatroban, bivalirudin, hirudin, etc.). For another example, a protein/molecule can be used to mimic thrombin to bind/sequester/inactivate heparin agents such as unfractionated heparin and enoxaparin. For a further example, a monoclonal antibody such as idarucizumab can bind to thrombin inhibitor dabigatran.

In some embodiments, the disclosed prosthetic comprises an agent that can reverse the effect of multiple anticoagulant. For example, ciraparantag (aka “PER977,” IUPAC name N1,N1′-[Piperazine-1,4-diylbis(propane-1,3-diyl)]bis-L-argininamide) and other similar molecular compounds can reverse multiple anticoagulant molecules such as rivaroxaban, apixaban, dabigatran, unfractionated heparin, and low molecular weight heparins.

In some embodiments, the disclosed prosthetic comprises proteins, antibodies, and other molecules that bind, block, inhibit, or sequester injected Factor Xa inhibitors (e.g. enoxaparin, dalteparin, fondaparinux, tinzaparin, etc.). Exemplary proteins or molecules can include protamine, ciraparantag, or anti-thrombin-like proteins. In some embodiments, the disclosed prosthetic comprises a lipid, such as a negatively charged phospholipid.

In some embodiments, the disclosed prosthetic comprises a small molecule inhibitors of uPA, tPA, and/or plasmin.

Prosthetic

The disclosed prosthetic can be modified directly on its surface. The disclosed prosthetic can also be placed within another prosthetic that has a surface modification. The modification to the surface of the prosthetic can be covalently or non-covalently linked, impregnated, or otherwise associated with an agent. In some embodiments, the modification to the surface of the prosthetic can be covalently or non-covalently linked, impregnated, or otherwise associated with fibrinogen, fibrinogen derivatives, fibrin, or a combination thereof. In some embodiments, the modification to the surface of the prosthetic can be covalently or non-covalently linked, impregnated, or otherwise associated with collagen or with collagen in addition to fibrinogen and/or fibrinogen derivatives and/or fibrin. In certain embodiments, the modification to the surface of the prosthetic may be covalently or non-covalently linked, impregnated, or otherwise associated with one or more of the following: agents that can cleave lysine off of fibrin and/or fibrinogen, fibrin cross linking proteins, direct or indirect inhibitors of fibrinolysis, lysine analogues, agents that can link platelets to fibrin and/or fibrinogen, platelets activating agents, thrombin or thrombin-like proteins, coagulation factors, or any combinations thereof. In certain embodiments, the modification to the surface of the prosthetic may be covalently or non-covalently linked, impregnated, or otherwise associated with one or more of the following: thrombin-activatable fibrinolysis inhibitor (TAFI), carboxypeptidase B (CPB), Factor XIII, alpha 2-antiplasmin (A2AP), alpha 2-macroglobulin (A2MG), plasminogen activator inhibitor-1 (PAI-1), plasminogen activator inhibitor-2 (PAI-2), aminocaproic acid, tranexamic acid, Von Willebrand Factor (vWF), arachidonic acid, thromboxane, thrombin, prothrombin, venom from snakes of the Bothrops genus such as B. moojeni and B. atrox, Factors X, Factor Xa, Factor V, Factor Va, or any derivatives thereof.

In some embodiments, the disclosed prosthetic can further comprise a blood product. In some embodiments, the disclosed prosthetic can also be used in combination with a blood product. The blood product can be from a patient or non-patient subject, and can be plasma, platelet poor plasma, cryoprecipitate, fibrinogen, or platelets. The method of making the disclosed prosthetic containing the blood product can involve soaking, spraying, painting, dipping, or otherwise dispersing blood products on or in the prosthetic, or with other agents used to modify the prosthetic. The disclosed prosthetic containing a blood product can be used immediately in STOPS, or can be allowed to partially dry in to a gel-like substance before use, or can be dried further to become a solid substrate (e.g. lyophilized) before use.

In addition to the formation of any of the prosthetic embodiments above, the disclosed prosthetic can further comprise a dispersible agent. In certain embodiments, the dispersible agent can comprise at least one agent that can modify fibrin and/or fibrinogen and/or fibrinogen derivatives to render them more resistant to fibrinolysis (e.g. TAFI, CPB, etc.). In certain other embodiments, the dispersible agent can comprise an agent that can prevent formation of plasmin (e.g. PAI-1, tranexamic acid, etc.). In some other embodiments, the dispersible agent can comprise an agent that can inhibit plasmin (e.g. A2AP, A2MG, etc.), and/or prevent formation of plasmin-like proteins, and/or inhibit plasmin-like proteins. As used herein, plasmin-like proteins can refer to proteins that have similar function to plasmin under normal physiological conditions, and cause breakdown of fibrin and/or fibrinogen and/or fibrinogen derivatives. In a preferred embodiment, the dispersible agent can comprise an agent to inhibit the process of fibrinolysis, either directly or indirectly or both, if such a fibrinolysis inhibiting agent is not included in the prosthetic already. In certain embodiments, the dispersible agent can comprise some form of thrombin and/or prothrombin and/or other derivatives of prothrombin and/or thrombin-like protein (e.g. venom from snakes of the Bothrops genus like B. moojeni, B. atrox, etc.). As used herein, thrombin-like proteins can refer to proteins that have similar function to thrombin and can cleave fibrinogen (or fibrinogen fragments or derivatives) to fibrin and/or otherwise cause fibrin and/or fibrin-like proteins to polymerize and/or link together in a covalent and/or non-covalent way. In the above mentioned embodiments, blood products (e.g. plasma, platelet poor plasma, cryoprecipitate, fibrinogen, etc.) can be added to, or in addition to, the dispersible agent embodiments before, during, and/or after their application to the surgically created potential space.

In some particularly useful embodiments, one or more of the dispersible agents and/or the prosthetic can include one of the following agent: some form of thrombin, prothrombin, derivatives of prothrombin, thrombin-like protein (e.g. venom from snakes of the Bothrops genus like B. moojeni, B. atrox, etc.), platelets to supply thrombin, platelet products to supply thrombin that can cleave fibrinogen (or fibrinogen fragments or derivatives) to fibrin and/or otherwise cause fibrin and/or fibrin-like proteins to polymerize and/or link together in a covalent and/or non-covalent way. Additionally, in particularly useful embodiments, the disclosed dispersible agents and/or the prosthetic can be dispersed or placed in to the surgically created potential space at the time when the medical provider is ready to eliminate the surgically created potential space, which can cause fibrin clot formation and/or fibrin polymerization and can rapidly eliminate the surgically created potential space.

The agents used to modify the surface of the prosthetic and/or used in the dispersible agent can be derived in part or in whole, or any combination thereof, of recombinant proteins, modified recombinant proteins, native proteins, and/or modified native proteins, with or without pharmacologic agents.

In some embodiments, the STOPS method can involve using a prosthetic containing fibrinogen and/or fibrinogen derivatives and/or fibrin covalently and/or non-covalently linked and/or impregnated in and/or otherwise associated with the prosthetic surface in combination with one or more dispersible agents. The composition containing the modified prosthetic and one or more dispersible agents can comprise one of the following agents: collagen, TAFI, CPB, Factor XIII, A2AP, A2MG, PAI-1, PAI-2, lysine analogues (such as aminocaproic acid and tranexamic acid), thrombin, prothrombin, derivatives of prothrombin, thrombin-like protein, vWF, platelet activating agents (e.g. arachidonic acid, thromboxane, etc.), Factor XIII, or any combinations thereof.

The agents used to modify the surface of the prosthetic and/or used in the dispersible agent can be present in different concentrations or be combined in different ratios. In some embodiments, the disclosed prosthetic can comprise fibrinogen and/or fibrinogen derivatives and/or fibrin that is close to physiological concentrations. In some embodiments, the disclosed prosthetic can further comprise thrombin, prothrombin, derivatives of prothrombin, or thrombin-like protein that are at or above physiological concentrations. In some embodiments, the concentrations of thrombin, prothrombin, derivatives of prothrombin, or thrombin-like protein can be at least 1-fold, at least 2-fold, at least 3-fold, at least 4-fold, at least 5-fold, at least 6-fold, at least 7-fold, at least 8-fold, at least 9-fold, or at least 10-fold than the physiological concentrations. In some embodiments, the disclosed prosthetic can further comprise a plasmin inhibitor that is at a concentration higher than the concentration of fibrinogen/fibrin, or the concentration of thrombin/prothrombin. The concentration of the plasmin inhibitor can be at least 2-fold, at least 3-fold, at least 4-fold, at least 5-fold, at least 6-fold, at least 7-fold, at least 8-fold, at least 9-fold, or at least 10-fold more than the concentration of fibrinogen/fibrin, or the concentration of thrombin/prothrombin.

When performing the STOPS method, a prosthetic or multiple prosthetics generated as described herein can be placed in the surgically created potential space before, during, and/or after the dispersible agent described above. When this is done in certain embodiments, one or more first prosthetic (e.g. a port, or pacemaker, or AICD) may be placed within a second prosthetic (e.g. an absorbable mesh), wherein the first or the second or both can have surface modifications or impregnations. In some embodiments, the modifications/impregnations can be done before, during, and/or after their placement in the surgically created potential space. One or more of these prosthetics may, in certain embodiments, be mechanically, physically, chemically, biochemically and/or otherwise fixed to the surrounding body tissues. Exemplary methods to fix a prosthetic can include using synthetic or biologic glues, staples, sutures, or tacks to fix a fibrinogen-containing surface-modified mesh to the perimeter of the potential space. When all portions of the particular STOPS embodiment are present in the surgically created potential space in the desired fashion, including the prosthetics and/or dispersible agents that contain thrombin, thrombin-like protein (e.g. venom from snakes of the Bothrops genus like B. moojeni, B. atrox, etc.), prothrombin or its derivatives, and/or other sources of thrombin (e.g. platelet products) to the wound, the wound can then be physically held closed by a medical provider. Various methods/tools can be used to close the wound. Examples include, but are not limited to, surgical graspers, hooks, forceps, robot, sutures, staples, tacks, synthetic, biologic glue, and strips of tape. In some embodiments, the wound can be allowed to simply remain closed with the intrinsic wound integrity provided by STOPS, and firm pressure can be applied directly to the surgical site for a period of time by the medical provider or other assistant. In some embodiments, the period of time for applying the firm pressure can be at most 1 min, at most 2 min, at most 3 min, at most 4 min, at most 5 min, at most 6 min, at most 7 min, at most 8 min, at most 9 min, at most 10 min, at most 11 min, at most 12 min, at most 13, at most 14 min, at most 15 min, at most 16 min, at most 17 min, at most 18 min, at most 19 min, or at most 20 min. In some embodiments, the period of time for the firm pressure can be from 1 to 10 min, from 10 min to 20 min, from 20 min to 30 min, or from 30 min to 60 min. Application of STOPS followed by wound closure can result in the formation of fibrin clots that link together the prosthetic and opposing tissue (or opposing prosthetic that is fixed to the surrounding/opposing tissue), formation of a hemostatic biochemical link (i.e. a fibrin clot) that is resistant to breakdown or fibrinolysis due to the included fibrin modifiers (e.g. TAFI, factor XIII, etc.) and/or anti-fibrinolytic proteins/agents (e.g. A2AP, PAI-1, tranexamic acid, etc.), thereby durably preventing accumulation of fluid in the surgically created potential space. This method can be applicable to a number of specific surgical and procedural interventions currently in wide practice that suffer complications related to fluid accumulation (e.g. seroma, hematoma) in surgically created potential spaces, such as hernia repair with mesh, fascial closure with mesh, abdominal wall reconstruction with mesh, breast reconstruction with mesh, vascular graft placement or formation (including artery to artery, artery to vein, and/or vein to vein), vascular access “port” placement (commonly referred to as a “portacath”), pacemaker placement, CIED placement, etc.

Material and Device

The disclosed prosthetic used in STOPS method can be a material, a mesh or mesh-like material, a scaffold or scaffold-like material, or a device. In some embodiments, the material can be a film, a sheet, a membrane, a patch, or a combination thereof. The material can be porous or non-porous. The material can be absorbable, non-absorbable, or a combination thereof. In some embodiments, the material can be biologic or biologic-based, synthetic, biosynthetic, or a combination thereof. Exemplary synthetic material includes, but not limited to, polypropylene, polytetrafluoroethylene, polyglactin 910, and poliglecaprone 25. Exemplary biologic or biologic based material can include materials made from dermis, pericardium, intestinal wall, collagen, fibrinogen, fibrinogen derivative, fibrin, or a combination thereof. Exemplary biosynthetic material can include mixtures of polyglycolic acid and trimethylene carbonate, or mixtures of polyglycolic acid, polylactic acid, and trimethylene carbonate.

In some embodiments, the disclosed prosthetic can be a device. Exemplary device can include an implanted venous access catheter, vascular graft, port, pacemaker, CIED, orthopedic implants such as hip/knee replacements and spine hardware. Furthermore, examples of implantable devices that can be used with the present invention include self-expandable stents, balloon-expandable stents, stent-grafts, grafts (e.g., aortic grafts), vascular grafts, artificial heart valves, cerebrospinal fluid shunts, pacemaker electrodes, guidewires, ventricular assist devices, artificial hearts, cardiopulmonary by-pass circuits, blood oxygenators, and endocardial leads (e.g., FINELINE and ENDOTAK, available from Guidant Corporation, Santa Clara, Calif.). The underlying structure of the device can be of virtually any design. The device can be made of a metallic material or an alloy such as, but not limited to, cobalt chromium alloy (ELGILOY), stainless steel (316 L), high nitrogen stainless steel, e.g., BIODUR 108, cobalt chrome alloy L-605, “MP35N”, “MP20N”, ELASTINITE (Nitinol), tantalum, nickel-titanium alloy, platinum-iridium alloy, gold, magnesium, or combinations thereof “MP35N” and “MP20N” are trade names for alloys of cobalt, nickel, chromium, and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention.

In some embodiments, the disclosed prosthetic can be a mesh or mesh-like material. A mesh in accordance with the current disclosure can be any web or fabric with a construction of knitted, braided, woven or non-woven filaments or fibers that are interlocked in such a way to create a fabric or a fabric-like material. Surgical meshes are well known in the art and any such mesh can be modified as described herein. The meshes used in the current disclosure can be made from biocompatible materials, synthetic or natural, including but not limited to, polypropylene, polyester, polytetrafluroethylene, polyamides and any combinations thereof. The surface modifications can be used with any commercially available mesh. For example, a mesh can be made from woven polypropylene. Pore sizes of meshes vary. For example the Bard Marlex® mesh has pores of 379+/−143 micrometers or approx. 0.4 mm, whereas the Johnson and Johnson Vypro® mesh has pores of 3058+/−62 micrometers or approx. 3 mm.

In some embodiments, the prosthetic can be a mesh, and the mesh can be made radially elastic. In some embodiments, the mesh/material can be engineered to hold Young's modulus constant throughout its substance, but vary the thickness so that the overall radial spring constant drops off with distance from the center. Alternatively, the geometric stiffness of the fibers can be altered along a radius by varying their cross-sectional shape, without changing the weave. In a non-woven mesh, the fiber deposition isometry can be varied such that fibers are radially aligned near the center and grow more randomly aligned toward the edge. If the mesh is made of fibers of 2 materials or 2 cross-sectional profiles, the blend ratio can vary from center to edge. To provide a strong and pliable/elastic mesh, several strategies can be applied, including controlling elasticity of mesh across its contour, microscopically texturing the mesh such that interwoven fibers can slip and stretch amongst themselves, and designing the two sides of the mesh such that the two sides of the mesh can slip/slide with respect to each other.

In another aspect of the present invention, a prosthetic for implantation at a wound site comprises a self-expanding mesh having a collapsed configuration and an expanded configuration. The collapsed configuration is adapted to be delivered to the wound site, and the expanded configuration is adapted to expand the prosthetic into engagement with the wound site. The mesh in the expanded configuration can be personalized to conform to or otherwise match the wound site.

The self-expanding mesh may comprise a nitinol mesh, or it may comprise one or more filaments in a helical pattern. The mesh may also comprise barbs or hooks adapted to engage tissue at the treatment site and anchor the prosthetic. The mesh may also comprise a plurality of overlapping filaments forming twisted or overlapping regions. The overlapping regions form raised surfaces that may be adapted to engage tissue at the treatment site and anchor the prosthesis. The one or more filaments may be woven together to form overlapping regions with the filaments overlapping one another at least once, twice, three times, or more. In other embodiments, some of the overlapping regions may have a first number of overlaps of the filaments while in other overlapping regions there may be a second number of overlaps different than the first number.

In various embodiments, one or more agents can be coated onto the surface of the prosthetic to form a coating layer. The coating layer may be formed of any material capable of releasing the one or more agents into tissue when implanted in a subject. Preferably, the coating layer is suitable for at least temporary use within a human body. The coating layer is also preferably compatible with the agent. Examples of commonly used materials that may be used to form coating layers include polymers such as silicones, polyamines, polystyrene, polyurethane, acrylates, polysilanes, polysulfone, methoxysilanes, and the like. Any suitable polymeric material may be utilized. In some embodiments, the polymeric material of a coating is biodegradable. Coating layers may include or be formed from polymeric materials designed to control the rate at which the agent is released from the polymeric material. Any known or developed technology may be used to control the release rate. For example, a coating layer may be designed according to the teachings of WO/04026361, entitled “Controllable Drug Releasing Gradient Coating for Medical Devices.”

The modified prosthetic can be biocompatible and/or biodegradable. In addition, the modified prosthetic can be configured such that they do not interfere with any metabolic pathways that would produce significant biologic dysfunction. The use of sterile materials and components to form certain embodiments of the modified prosthetic can reduce or eliminate the risk of bacterial, viral, or other infectious agents being transmitted as the result of the use of the prosthetic. Certain embodiments of the modified prosthetic described herein can be prepared quickly and easily.

In addition, the components used to make certain embodiments of the prosthetic can have a relatively long shelf life, especially when enclosed in a sterile package.

Polymer and Additives

In some embodiments, the disclosed agent can be mixed into a polymer, or can be formed as part of a matric with a polymer. In some examples, the polymer is a biocompatible polymer. In some examples, biocompatible polymer may be biodegradable or bioabsorbable and be absorbed by the body of a subject after implantation of the disclosed prosthetic. In other examples, the polymer is not biodegradable and may remain in the body of the subject after implantation. In one example, polymer comprises a biocompatible and hydrophilic material that provides for relatively high loading of the modifying agent, relatively high stability of the modifying agent, and relatively fast release of the modifying agent. In some examples, polymer provides a suitable dispersion medium for the modifying agent so that the modifying agent may be maintained in a substantially homogenous dispersion for a predetermined period of time via blend uniformity and stability. In some examples, polymer may be bioabsorbable and/or biodegradable within the tissue and/or fluids of a subject, such that, in some examples, polymer, and hence the modifying agent, is completely released within a predetermined time frame. The polymer can be configured to maximize other properties, such as loading capacity of the modifying agent and stability of the modifying agent.

Examples of biocompatible hydrophilic materials that may be used for polymer of the current disclosure include, but are not limited to, polyvinylpyrrolidone (PVP), glycerol, polyethylene glycol (PEG), methyl polyethylene glycol, polyacrylic acid (PAA), polymethacrylic acid, polylactic acid (PLA), lactic acid, poly(lactic-co-glycolic acid) (PLGA), polycaprolactam, poly(trimethylene carbonate) (PMTC), chitosan, sucrose acetate isobutyrate (SAM), polyhydroxylalkanoate (PHA), polyhydroxybutyrate (PHB), carboxymethylchitosan-oxidized starch, poloxamers, polymethyl vinyl ether/maleic anhydride, and pluonics such as a polyethylene glycol-polypropylene glycol-polyethylene glycol (PEG-PPG-PEG) pluonic and/or a polypropylene glycol-polyethylene glycol-polypropylene glycol (PPG-PEG-PPG) pluonic. In some examples, N-methylpyrrolidone (NMP) and/or dimethyl sulfoxide (DMSO) may be able to be used to help make biodegradable polymers, such as PLGA into gels.

Other biocompatible polymers that may be used may include, for example, a polyurethane or a silicone. In some examples, the silicone or polyurethane may be mixed with hydrophilic polymer, such as the hydrophilic polymers described above, to provide a controlled release mechanism, e.g., because the silicone or polyurethane may slow down the release of the modifying agent. Various types of silicone may be used, including, for example, silicone pressure sensitive adhesive (PSA), room temperature vulcanization (curing) (RTV) silicone, liquid silicone rubber (LSR), enhanced tear resistance (ETR) silicone, or the like. Exemplary silicones include, but are not limited to, Silastic® Q-7-4850 LSR, available from Dow Corning, Corp., Midland, Mich.; Silastic® MDX4-4210, available from Dow Corning, Corp., Midland, Mich.; Q7-4735, Q7-4750, and Q7-4765 ETR silicones, available from Dow Corning, Corp., Midland, Mich.; NuSil MED-1137 and NuSil MED-200 RTV silicones, available from NuSil Technology, LLC, Carpinteria, Calif.; Rehau SI-1511 RTV silicone, available from Rehau Co., Leesburg, Va.; Silastic® MDX7-4502, BIO-PSA 7-4501, BIO-PSA 7-4402, BIO-PSA 7-4502, BIO-PSA-4602, 7-9800 SSA, and MG7-9850 PSA silicones, available from Dow Corning, Corp., Midland, Mich. In some examples, the at least one modifying agent may be mixed into the silicone or a constituent of the silicone, prior to curing, while in other embodiments, the at least one modifying agent may be mixed into the silicone subsequent to curing of the silicone.

In other examples, polymer can comprise another PSA, such as an acrylic PSA, a polyisobutylene PSA, a polyurethane PSA, a cyanoacrylate PSA, a PLGA-based PSA, or the like. In examples such as these, the at least one modifying agent may be mixed into the PSA.

In other examples, the biocompatible polymer may also include a biodegradable or bioabsorbable polymer, such as, for example, collagen, poly(lactic-co-glycolic acid) (PLGA), poly(lactic acid) (PLA), poly(glycolic acid) (PGA), polyethylene glycol (PEG), PEG stearate, poly(ethylene oxide) (PEO), poly(ethylene co-vinyl acetate), poly(ortho ester) (POE), poly(ε-caprolactone) (PCL), poly(dioxanone), polyglyconate, hyaluronic acid, gelatin, fibrin, fibrinogen, cellulose, starch, cellulose acetate, polyvinylpyrrolidone (PVP), a poly(ethylene oxide)/poly(propylene oxide) copolymer (PEO-PPO), a polyethylene-polypropylene glycol copolymer, poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as PEO/PLA, a polyalkylene oxalate, a polyphasphazene, a polyarylate, a tyrosine-based biodegradable or bioabsorbable polymer, poly hydroxyalkanoate (PHA), poloxamers, polymethyl vinyl ether/maleic anhydride, a sugar ester, or the like. In some embodiments, the disclosed prosthetic can comprise a tyrosine polyarylate. The biodegradable or bioabsorbable polymer may degrade and be absorbed by the body of a subject over time after implantation of the prosthetic in the body of the subject. This may be advantageous because it may ensure that substantially all the modifying agent is released, which may reduce risk of the growth or development of organisms that are resistant to the modifying agent. Further, absorption of the polymer over time may remove a site at which bacteria can grow.

Additional exemplary polymer can include polycaprolactone, poly(D,L-lactide), poly(L-lactide), poly(D,L-lactide-co-L-lactide), poly(glycolide), poly(D,L-lactide-co-glycolide), poly(dioxanone), poly(4-hydroxybutyrate), poly(3-hydroxybutyrate), poly(3-hydroxy valerate), poly(hydroxybutyrate-co-hydroxyvalerate), poly(tyrosine derive carbonates), poly(tyrosine arylates), poly(imino carbonates), poly(trimethylene carbonate), poly(anhydrides), poly(orthoesters), and poly(ester amides).

The disclosed prosthetic may include other components that may influence the properties of the prosthetic. For example, the disclosed prosthetic may include an additive that influences the release rate of the antimicrobial, such as a plasticizer or another excipient. In another example, a surfactant may be added to the prosthetic, which may allow for higher loading of modifying agent. A plasticizer or excipient may affect the viscosity of the polymer, which may in turn affect the release rate of the at least one antimicrobial. Thus, incorporation of a plasticizer or excipient may be one manner in which the time over which the antimicrobial is released from reservoir body 48 is affected. In some examples, the additive may swell or dissolve in biological fluids present in the body of a subject, which may affect the release rate of the antimicrobial. Exemplary additives that influence the release rate of the modifying agent may include, for example, poly(acrylic acid), poly(methacrylic acid), poly(vinylpyrolidone), a sugar ester, macrogol 15 hydroxystearate (IV), poly(lactic acid), lactic acid, glycerol, poly(ethylene glycol) (PEG), methyl polyethylene glycol (methyl PEG), poly(glycolic acid), poly(ε-caprolactum), polysorbate 80 (polyoxyethylene (20) sorbitan monooleate), polysorbate 20 (polyoxyethylene (20) sorbitan monolaurate), salts such as KCl, cationic surfactants, anionic surfactants, natural surfactants, or the like. Exemplary surfactants include, but are not limited to, sodium dodecyl sulfate (SDS), sodium stearate, sucrose stearate, stearyl alcohol, glycerol monostearate, mannitol, sodium laureth sulfate, sodium lauryl sulfate, triton X 100, sorbitol, fructose, chitosan, hyaluronic acid, alginate, and trimethyldodecylammonium (TMDA). As another example, the disclosed prosthetic may include fumed silica. Fumed silica may increase polymer integrity, such as integrity of a silicone PSA, and may also facilitate faster release of the modifying agent. In some examples, the additive that influences the release rate of the modifying agent from the prosthetic may constitute less than approximately 1 weight percent (wt. %) of the prosthetic.

In some examples, the disclosed prosthetic can further comprise a biosoluble material, such as hydrophilic excipient, which may comprise at least one of polyvinylpyrolidone (PVP), polyacrylic acid (PAA), polymethacrylic acid, poly(lactic-co-glycolic acid) (PLGA), polylactic acid (PLA) or its monomer lactic acid, polyethylene glycol (PEG), polycaprolactam, methyl polyethylene glycol (methyl PEG), poly(glycolic acid) (PGA), poly(ethylene oxide) (PEO), poly(ortho ester) (POE), poly(ε-caprolactone) (PCL), poly(dioxanone), polyglyconate, a polyvinylalcohol, a poly(ethylene oxide)/poly(propylene oxide) copolymer (PEO-PPO), poly(ethylene vinyl acetate), poly(hydroxybutyrate-covalerate), polyanhydride, poly(glycolic acid-co-trimethylene carbonate), polyphosphoester, polyphosphoester urethane, a poly(amino acid), a cyanoacrylate, poly(trimethylene carbonate), poly(iminocarbonate), a copoly(ether-ester) such as PEO/PLA, a polyalkylene oxalate, a polyphasphazene, a polyarylate, a tyrosine-based biodegradable or bioabsorbable polymer, poly hydroxyalkanoate (PHA), a sugar ester, hyaluronic acid, macrogol 15 hydroxystearate (IV), glycerol, polyglycolic acid, poly(ε-caprolactum), polysorbate 80 (polyoxyethylene (20) sorbitan monooleate), polysorbate 20 (polyoxyethylene (20) sorbitan monolaurate), collagen, gelatin, fibrin, fibrinogen, cellulose, starch, cellulose acetate, or the like.

Applications

The modified prosthetic described herein can be used in a wide variety of applications including, for example, general surgery, vascular surgery, spine surgery and ophthalmologic surgery. The modified prosthetic described herein can be configured to be applied to any type of tissue including soft tissue, bone tissue, or any other type of tissue. The modified prosthetic can be employed to: assist hemostasis in a bleeding area, reduce blood flow from solid organs, assist in sealing suture holes, assist in sealing anastomosis or leaks from hollow organs, assist or replace sutures in surgical procedures (particularly where suturing is difficult or impossible), produce a water-tight closure across portions of tissue (e.g., across a suture line), reinforce tissue (e.g., in reinforcing suture lines including high stress suture lines), perform of tissue approximation, replace sutures, fill dead space or other voids in tissue, and/or in vascular repair (e.g., to seal a vascular defect). In certain embodiments, the modified prosthetic described herein can be employed to perform gastrointestinal suture line reinforcement, in preventing the formation of seroma (e.g., after surgical procedures), for use as soft tissue (e.g., after breast cancer or other surgical procedures in which tissue may be removed), as burn dressings, and/or for combined hemostasis/sealing and drug delivery.

In some embodiments, the modified prosthetic can be used to treat spleen tissue, for example, to inhibit or stop bleeding or the leaking of other bodily fluids and/or to partially or completely fill void(s) in the spleen. In certain embodiments, the modified prosthetic can be used to treat lung tissue, for example, to inhibit or stop bleeding or the leaking of other bodily fluids, to partially or completely fill void(s) in the lung, and/or to inhibit or stop the leaking of air from the internal cavity of a lung. In some embodiments, the modified prosthetic described herein can be used to treat the liver, for example, to inhibit or stop bleeding or the leaking of other bodily fluids from the liver and/or to partially or completely fill void(s) in the liver. In certain embodiments, the modified prosthetic can be used to treat heart tissue, for example, to inhibit or stop bleeding or the leaking of other bodily fluids, to partially or completely fill void(s) in the heart or associated blood vessels, and/or to inhibit or stop the leaking of blood from an internal cavity of a heart. The modified prosthetic described herein can also be used to treat tissues in or near the gastrointestinal tract, for example, to inhibit or stop bleeding or the leaking of other bodily fluids, to partially or completely fill void(s) in gastrointestinal tissues.

Subjects

Subjects can be, for example, mammal, humans, pregnant women, elderly adults, adults, adolescents, pre-adolescents, children, toddlers, infants, newborn, or neonates. A subject can be a patient or a non-patient. In some cases, a subject can be a human. In some cases, a subject can be a child (i.e. a young human being below the age of puberty). In some cases, a subject can be an infant. In some cases, the subject can be a formula-fed infant. In some cases, a subject can be an individual enrolled in a clinical study. In some cases, a subject can be a laboratory animal, for example, a mammal, or a rodent.

The modified prosthetic described herein can be used to treat human subjects, in certain embodiments. In other embodiments, the modified prosthetic described herein can be used to treat non-human animal subjects. For example, in certain cases, the modified prosthetic described herein can be used in veterinary applications, for example, those involving horses, dogs, cats, and the like.

In certain embodiments, the disclosed invention can be used to treat a subject that is under anticoagulant treatment or has been treated with anticoagulant. Examples of anticoagulants can include low molecular weight heparins such as enoxaparin)(LOVENOX®, dalteparin (FRAGMIN®) and tinzaparin (INNOHEP®); heparin; heparinoids such as danaparoid (ORGARAN®); pentasaccharides such as fondaparinux (ARIXTRA®); as well as argatroban, warfarin) (COUMADIN® and rivaroxaban)(XARELTO®). In such situations, anticoagulant antagonists or devices may be used in combination with the disclosed prosthetic. For example, divalent antibody or modified antibody that targets heart endothelium and binds to the anticoagulant can localize anticoagulation to an organ of interest. For another example, divalent antibody that targets TF or collagen IV (COL4) or other basement membrane proteins can sequester anticoagulant when the anticoagulant is delivered topically or intravenous. For another example, artificial cardiac valves can be bound with a targeted anticoagulant to avoid systemic anticoagulation.

Combinations

The prosthetic disclosed herein can further comprise other agents that may be beneficial for wound healing. Examples of agents suitable for use with STOPS include anesthetics, antibiotics (antimicrobials), anti-inflammatory agents, fibrosis-inhibiting agents, anti-scarring agents, leukotriene inhibitors/antagonists, cell growth inhibitors and the like. As used herein, agent can include all types of therapeutic agents or drugs, whether small molecules or large molecules such as proteins, nucleic acids and the like. Those of skill in the art can readily determine the amount of a particular agent to include in the surface modification of the prosthetic disclosed herein.

Any pharmaceutically acceptable form of the drugs can be employed in the STOPS of the current disclosure, e.g., the free base or a pharmaceutically acceptable salt or ester thereof. Pharmaceutically acceptable salts, for instance, include sulfate, lactate, acetate, stearate, hydrochloride, tartrate, maleate, citrate, phosphate and the like. In certain embodiments, a pain control agent or anesthetic agent (e.g. delayed-release liposomal bupivacaine, bupivacaine, lidocaine, xylocaine, etc.) can be covalently and/or non-covalently linked and/or otherwise associated with and/or impregnated in the prosthetic, and/or included in one or more of the dispersible agents, and/or added separately from any of the other steps in performing any of the embodiments of STOPS. In certain embodiments, antibiotics (e.g. bacterial cell wall synthesis inhibitors such as vancomycin or penicillins and their derivatives, aminoglycosides such as gentamicin, fluoroquinolones such as ciprofloxacin, macrolides such as erythromycin, other ribosomal inhibitors such as tetracyclines, etc.) can be covalently and/or non-covalently linked to and/or impregnated in and/or otherwise associated with the prosthetic, and/or included in one or more of the dispersible agents, and/or added separately from any of the other steps in performing any of the embodiments of STOPS.

Examples of non-steroidal anti-inflammatories include, but are not limited to, naproxen, ketoprofen, ibuprofen as Well as diclofenac; celecoxib; sulindac; diflunisal; piroxicam; indomethacin; etodolac; meloxicam; r-flurbiprofen; mefenamic; nabumetone; tolmetin, and sodium salts of each of the foregoing; ketorolac bromethamine; ketorolac bromethamine tromethamine; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, l, and racemic isomers); and the methyl, ethyl, propyl, isopropyl, n-butyl, sec-butyl, t-butyl, esters of acetylsalicylic acid.

Examples of anesthetics include, but are not limited to, licodaine, bupivacaine, and mepivacaine. Further examples of analgesics, anesthetics and narcotics include, but are not limited to acetaminophen, clonidine, benzodiazepine, the benzodiazepine antagonist flumazenil, lidocaine, xylocaine, tramadol, carbamazepine, meperidine, zaleplon, trimipramine maleate, buprenorphine, nalbuphine, pentazocain, fentanyl, propoxyphene, hydromorphone, methadone, morphine, levorphanol, and hydrocodone.

Examples of antimicrobials include, but are not limited to, triclosan, chlorhexidine, rifampin, minocycline, vancomycin, gentamycine, cephalosporins and the like. In some embodiments, the surface modification contains rifampin and another anti-microbial agent. In some other embodiments, the surface modification contains a cephalosporin and another antimicrobial agent. Preferred combinations can include rifampin and minocycline, rifampin and gentamycin, and rifampin and minocycline.

Further antimicrobials include aztreonam; cefotetan and its disodium salt; loracarbef; cefoxitin and its sodium salt; cefazolin and its sodium salt; cefaclor; ceftibuten and its sodium salt; ceftizoxime; ceftizoxime sodium salt; cefoperazone and its sodium salt; cefuroxime and its sodium salt; cefuroxime axetil; cefprozil; ceftazidime; cefotaxime and its sodium salt; cefadroxil; ceftazidime and its sodium salt; cephalexin; cefamandole nafate; cefepime and its hydrochloride, sulfate, and phosphate salt; cefdinir and its sodium salt; ceftriaxone and its sodium salt; cefixime and its sodium salt; cefpodoxime proxetil; meropenem and its sodium salt; imipenem and its sodium salt; cilastatin and its sodium salt; azithromycin; clarithromycin; dirithromycin; erythromycin and hydrochloride, sulfate, or phosphate salts ethylsuccinate, and stearate forms thereof; clindamycin; clindamycin hydrochloride, sulfate, or phosphate salt; lincomycin and hydrochloride, sulfate, or phosphate salt thereof; tobramycin and its hydrochloride, sulfate, or phosphate salt; streptomycin and its hydrochloride, sulfate, or phosphate salt; vancomycin and its hydrochloride, sulfate, or phosphate salt; neomycin and its hydrochloride, sulfate, or phosphate salt; acetyl sulfisoxazole; colistimethate and its sodium salt; quinupristin; dalfopristin; amoxicillin; ampicillin and its sodium salt; clavulanic acid and its sodium or potassium salt; penicillin G; penicillin G benzathine, or procaine salt; penicillin G sodium or potassium salt; carbenicillin and its disodium or indanyl disodium salt; piperacillin and its sodium salt; ticarcillin and its disodium salt; sulbactam and its sodium salt; moxifloxacin; ciprofloxacin; ofloxacin; levofloxacins; norfloxacin; gatifloxacin; trovafloxacin mesylate; alatrofloxacin mesylate; trimethoprim; sulfamethoxazole; demeclocycline and its hydrochloride, sulfate, or phosphate salt; doxycycline and its hydrochloride, sulfate, or phosphate salt; minocycline and its hydrochloride, sulfate, or phosphate salt; tetracycline and its hydrochloride, sulfate, or phosphate salt; oxytetracycline and its hydrochloride, sulfate, or phosphate salt; chlortetracycline and its hydrochloride, sulfate, or phosphate salt; metronidazole; dapsone; atovaquone; rifabutin; linezolide; polymyxin B and its hydrochloride, sulfate, or phosphate salt; sulfacetamide and its sodium salt; and clarithromycin.

Examples of antifungals include amphotericin B; pyrimethamine; flucytosine; caspofungin acetate; fluconazole; griseofulvin; terbinafin and its hydrochloride, sulfate, or phosphate salt; ketoconazole; micronazole; clotrimazole; econazole; ciclopirox; naftifine; and itraconazole.

Other drugs that can be incorporated into the prosthetic include, but are not limited to, keflex, acyclovir, cephradine, malphalen, procaine, ephedrine, adriamycin, daunomycin, plumbagin, atropine, quinine, digoxin, quinidine, biologically active peptides, cephradine, cephalothin, cis-hydroxy-L-proline, melphalan, penicillin V, aspirin, nicotinic acid, chemodeoxycholic acid, chlorambucil, paclitaxel, 5-flurouracil and the like.

Examples of useful proteins include cell growth inhibitors such as epidermal growth factor.

Examples of anti-inflammatory compound include, but are not limited to, anecortive acetate; tetrahydrocortisol,4,9(11)-pregnadien-17.alpha.,21-diol-3,20-dione and its -21-acetate salt; 11-epicortisol; 17.alpha.-hydroxyprogesterone; tetrahydrocortexolone; cortisona; cortisone acetate; hydrocortisone; hydrocortisone acetate; fludrocortisone; fludrocortisone acetate; fludrocortisone phosphate; prednisone; prednisolone; prednisolone sodium phosphate; methylprednisolone; methylprednisolone acetate; methylprednisolone, sodium succinate; triamcinolone; triamcinolone-16,21-diacetate; triamcinolone acetonide and its -21-acetate, -21-disodium phosphate, and -21-hemi succinate forms; triamcinolone benetonide; triamcinolone hexacetonide; fluocinolone and fluocinolone acetate; dexamethasone and its 21-acetate, -21-(3,3-dimethylbutyrate), -21-phosphate disodium salt, -21-diethylaminoacetate, -21-isonicotinate, -21-dipropionate, and -21-palmitate forms; betamethasone and its -21-acetate, -21-adamantoate, -17-benzoate, -17,21-dipropionate, -17-valerate, and -21-phosphate disodium salts; beclomethasone; beclomethasone dipropionate; diflorasone; diflorasone diacetate; mometasone furoate; and acetazolamide.

In any embodiments described herein, any of the components or agents of the device/prosthetic, for example, fibrin or fibrinogen, can be dispersible.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An device comprising:
a) a material with a surface comprising a modified fibrinogen or a modified fibrin, wherein the modified fibrinogen or modified fibrin is more resistant to fibrinolysis than an unmodified fibrinogen or an unmodified fibrin, respectively; and
b) one or more dispersible fibrinolysis inhibitors in contact with the surface comprising a plasmin inhibitor; and
c) optionally, a thrombin agent or platelets.

2. An implantable or topically applicable device comprising:
a) a material with a surface comprising fibrinogen or fibrin attached thereto; and
b) one or more dispersible fibrinolysis inhibitors attached to the surface selected from the group consisting of:
i) a fibrin and/or fibrinogen modifying agent; and
ii) a plasmin inhibitor;
c) optionally, a thrombin agent and/or platelets.

3. The device of claim 1 or 2, wherein the modified fibrinogen or the modified fibrin is more resistant to plasminogen or plasmin cleavage than an unmodified fibrinogen or an unmodified fibrin, respectively.

4. The device of any one of claims 1-3, wherein the device is implantable.

5. The device of any one of claims 1-3, wherein the device is topically applicable.

6. The device of any one of claims 1-5, wherein the surface further comprises collagen.

7. The device of any one of claims 2-6, wherein the fibrin or fibrinogen modifying agent comprises an enzyme that cleaves the fibrin or fibrinogen.

8. The device of claim 7, wherein the fibrin or fibrinogen modifying agent comprises a thrombin-activatable fibrinolysis inhibitor (TAFI).

9. The device of claim 7, wherein the fibrin or fibrinogen modifying agent comprises a carboxypeptidase.

10. The device of claim 7, wherein the fibrin or fibrinogen modifying agent cleaves a C-terminal amino acid of the fibrin or fibrinogen.

11. The device of claim 7, wherein the fibrin or fibrinogen modifying agent cleaves a lysine of the fibrin or fibrinogen.

12. The device of any one of claims 1-11, wherein the plasmin inhibitor comprises a plasmin formation inhibitor.

13. The device of any one of claims 1-12, wherein the plasmin inhibitor comprises a plasmin activity inhibitor.

14. The device of any one of claims 1-13, wherein the plasmin inhibitor comprises a direct or indirect inhibitor of fibrinolysis.

15. The device of any one of claims 1-14, wherein the plasmin inhibitor comprises alpha 2-antiplasmin (A2AP), alpha 2-macroglobulin (A2MG), plasminogen activator inhibitor-1 (PAI-1), plasminogen activator inhibitor-2 (PAI-2)), a lysine analogue, or a combination thereof.

16. The device of claim 15, wherein the lysine analogue is aminocaproic acid or tranexamic acid.

17. The device of any one of claims 1-16, wherein the plasmin inhibitor comprises an inhibitor of uPA.

18. The device of any one of claims 1-17, wherein the plasmin inhibitor comprises an inhibitor of tPA.

19. The device of any one of claims 1-18, wherein the plasmin inhibitor comprises an inhibitor of streptokinase.

20. The device of any one of claims 1-19, wherein the plasmin inhibitor comprises an inhibitor of plasmin.

21. The device of any one of claims 1-20 wherein the plasmin inhibitor or a plasmin activity inhibitor agent is a small molecule.

22. The device of any one of claims 1-20, wherein the plasmin inhibitor or a plasmin activity inhibitor agent is an antibody.

23. The device of any one of claims 1-22 wherein the thrombin agent comprises thrombin, prothrombin, or derivatives thereof.

24. The device of any one of claims 1-23, wherein the thrombin agent comprises an inhibitor of TFPI, an inhibitor of Protein S, or an inhibitor of activated Protein C, such as a natural or recombinant protein or other pharmacologic agent.

25. The device of any one of claims 1-24, wherein the thrombin agent comprises alpha-thrombin.

26. The device of any one of claims 23-25, wherein the thrombin agent is a natural or recombinant protein that is resistant to inactivation by anti-thrombin, oral direct thrombin, such as dabigatran or efegatran, or injected thrombin inhibitors, such as argatroban, bivalirudin or hirudin

27. The device of any one of claims 1-26, wherein the device further comprises fibrin or fibrinogen linking agent.

28. The device of claim 27, wherein the fibrin or fibrinogen linking agent links the fibrin or fibringogen to platelets.

29. The device of claim 27, wherein the fibrin or fibrinogen linking agent comprises a Von Willebrand Factor (vWF).

30. The device of any one of claims 1-29, wherein the device further comprises a platelet activating agent.

31. The device of claim 30, wherein the platelet activating agent comprises arachidonic acid or thromboxane.

32. The device of claim 30, wherein the platelet activating agent comprises a coagulation factor.

33. The device of claim 32, wherein the coagulation factor comprises factor X, factor Xa, factor V, factor Va, factor XII, factor XIIa, factor XI, factor XIa, factor IX, factor IXa, factor VIIIa, Tissue Factor, an inhibitor of Activated Protein C, an inhibitor of Protein S, an inhibitor of an anticoagulant, or an inhibitor of injected factor Xa.

34. The device of any one of claims 30-33, wherein the platelet activating agent is a recombinant protein.

35. The device of claim 34, wherein the factor V is a R506Q variant, a R534Q variant, resistant to cleavage or inactivation by Activated Protein C, a variant with increased activity compared to natural factor V, or a constitutively active variant.

36. The device of claim 34, wherein the factor X or factor Xa is resistant to inactivation, a variant with increased activity compared to natural Factor X or Xa, or a constitutively active variant.

37. The device of claim 36, wherein the factor X or factor Xa is resistant to inactivation in the presence of a heparin agent, such as unfractionated heparin, enoxaparin, dalteparin, fondaparinux, or tinzaparin.

38. The device of claim 36, wherein the factor X or factor Xa is resistant to inactivation by an oral Factor Xa inhibitor, such as apixaban, rivaroxaban, betrixaban, darexaban, edoxaban, otamixaban, letaxaban, or eribaxaban.

39. The device of claim 34, wherein the factor VII or factor VIIa is resistant to inactivation, a variant with increased activity compared to natural VII or factor VIIa, a constitutively active variant or a variant with increased protease activity with or without association with Tissue Factor.

40. The device of claim 34, wherein the Tissue Factor is resistant to inactivation, a variant with increased activity compared to natural Tissue Factor, a constitutively active variant or a variant with increased protease activity.

41. The device of claim 34, wherein the factor VIII or factor VIIIa is a constitutively active variant, functions as a cofactor for Factor IXa independent of cleavage events, or a variant more resistant to inactivation, such as inactivation by Activated Protein C.

42. The device of claim 34, wherein the factor IX or factor IXa is a constitutively active variant, variant more resistant to inactivation, or a variant with increased protease activity with or without association with Factor VIIIa or similar derivatives.

43. The device of claim 34, wherein the inhibitor of Activated Protein C is a recombinant protein or other pharmacologic agent that inhibits activity of the Activated Protein C.

44. The device of claim 34, wherein the inhibitor of Protein S is an antibody, nanoparticle, or other pharmacologic agents that blocks, inhibits, or sequesters Protein S.

45. The device of claim 34, wherein the inhibitor of the anticoagulant is an antibody, nanoparticle, molecular decoy, decoy receptor, or other pharmacologic sequestration/binding agent that binds, blocks, inhibits, or sequesters oral anticoagulants or oral Factor Xa inhibitor, or a protein or molecule that mimics Factor II or Factor IIa to bind/sequester an oral direct thrombin inhibitor or an injected thrombin inhibitor.

46. The device of claim 45, wherein the inhibitor of the anticoagulant is apixaban, rivaroxaban, betrixaban, darexaban, edoxaban, otamixaban, letaxaban, eribaxaban, Andexanet alfa, rivaroxaban, apixaban, dabigatran, efegatran, argatroban, bivalirudin, hirudin, a protein or molecule that mimics anti-thrombin to bind/sequester/inactivate a heparin agent such as unfractionated heparin or enoxiparin, or a monoclonal antibody such as Idarucizumab that binds dabigatran.

47. The device of claim 34, wherein the inhibitor of the anticoagulant is ciraparantag (“PER977,” IUPAC name N¹,N^{1′}-[Piperazine-1,4-diylbis(propane-1,3-diyl)]bis-L-argininamide) or a compound that reverses anticoagulant molecules, such as rivaroxaban, apixaban, dabigatran, unfractionated heparin, and low molecular weight heparins.

48. The device of claim 34, wherein the inhibitor of the anticoagulant is an antibody, such as a divalent antibody, that targets a basement membrane protein such as TF or COL4, sequesters an anticoagulant, such as an anticoagulant delivered deliver topically or by IV.

49. The device of claim 34, wherein the inhibitor of the anticoagulant is an antibody, such as a divalent antibody or modified antibody that targets an organ's endothelium and contains an anticoagulant such that anticoagulation is localized to an organ of interest.

50. The device of claim 34, wherein the inhibitor of injected factor Xa is a protein, antibody, or other pharmacologic molecule that binds, blocks, inhibits, or sequesters injected Factor Xa inhibitors, such as enoxaparin, dalteparin, fondaparinux, tinzaparin, protamine, or an anti-thrombin-like protein.

51. The device of claim 34, wherein the coagulation factor comprises a lipid, such as a negatively charged phospholipid.

52. The device of claim 34, wherein the coagulation factor comprises aprotinin.

53. The device of any one of claims 30-33, wherein the platelet activating agent is a natural protein.

54. The device of any one of claims 1-53, wherein the device further comprises a fibrin cross-linking agent.

55. The device of claim 54, wherein the fibrin cross-linking agent comprises Factor XIII.

56. The device of any one of claims 1-55, wherein the device further comprises a pain control agent or anesthetic agent (e.g. delayed-release liposomal bupivacaine, bupivacaine, lidocaine, xylocaine, etc.)

57. The device of any one of claims 1-56, wherein the device further comprises an antibiotic, such as a bacterial cell wall synthesis inhibitor such as vancomycin or a penicillin or derivatives thereof, aminoglycosides such as gentamicin, fluoroquinolones such as ciprofloxacin, macrolides such as erythromycin, other ribosomal inhibitors such as tetracycline.

58. The device of any one of claims 1-57, wherein the device further comprises a patient blood product.

59. The device of any one of claims 1-58, wherein the fibrinogen or fibrin is attached to the surface covalently.

60. The device of any one of claims 1-58, wherein the fibrinogen or fibrin is attached to the surface noncovalently.

61. The device of any one of claims 1-60, wherein the one or more dispersible fibrinolysis inhibitors agents are recombinant proteins, modified recombinant proteins, native proteins, and/or modified native proteins, with or without pharmacologic agent(s).

62. The device of any one of claims 1-61, wherein the material is a film, sheet, patch, device, vascular graft, mesh, mesh-like material, or a scaffold-like material.

63. The device of claim 62, wherein the material is biologic, synthetic, biosynthetic or a combination of biologic and/or synthetic and/or biosynthetic origin.

64. The device of claim 62, wherein the material is polyester, polypropylene, polytetrafluoroethylene, polyglactin 910, or poliglecaprone 25.

65. The device of claim 62, wherein the material is an implanted venous access catheter or port, pacemaker, or CIED.

66. The device of claim 62, wherein the material is porous.

67. The device of claim 62, wherein the material is non-porous.

68. The device of claim 62, wherein the material is absorbable.

69. The device of claim 62, wherein the material is non-absorbable.

70. The device of claim 62, wherein the material is made from dermis, pericardium, intestinal wall, collagen, fibrinogen, fibrinogen derivative, or fibrin.

71. The device of claim 62, wherein the material is biosynthetic such as a mixture of polyglycolic acid and trimethylene carbonate or a mixture of polyglycolic acid, polylactic acid, and trimethylene carbonate, or another polymer mixture.

72. The device of any one of claims 62-71, wherein the material comprises minocycline, rifampin or both.

73. The device of claim 72, wherein the minocycline, rifampin or both is releasable from the material.

74. The device of any one of claims 1-73, wherein the implantable device is a material, device, mesh, mesh-like material, or scaffold.

75. The device of any one of claims 1-74, wherein the device is a mesh.

76. The device of any one of claims 1-75, wherein the device is elastic.

77. The device of any one of claims 1-76, wherein the device is radially elastic.

78. The device of any one of claims 1-77, wherein the device is an orthopedic implant.

79. The device of any one of claims 1-78, wherein the material has a substantially uniform Young's modulus constant throughout.

80. The device of claim 79, wherein a thickness of the material is varied, wherein an overall radial spring constant decreases with distance from the center.

81. The device of any one of claims 1-80, wherein the material comprises woven fibers, wherein a geometric stiffness of the fibers is varied along a radius of the material by varying a cross-sectional shape of the fibers without changing a weave of the fibers.

82. The device of any one of claims 1-80, wherein the material comprises an unwoven mesh of fibers, wherein a deposition isometry of the fibers is varied such that the fibers are radially aligned near a center of the material and/or increase in random alignment toward an edge of the material edge.

83. The device of any one of claims 1-82, wherein the material comprises a mesh made of fibers made of 2 or more substances or 2 or more cross-sectional profiles.

84. The device of claim 83, wherein a blend ratio of the mesh made of fibers varies from a center of the surface to an edge of the surface.

85. The device of any one of claims 1-84, wherein the material comprises a mesh and wherein an elasticity of the mesh across a contour is varied.

86. The device of any one of claims 1-85, wherein the material comprises a mesh made of fibers that is microscopically textured such that interwoven fibers are free to, move, slip, or stretch amongst themselves.

87. The device of any one of claims 1-86, wherein the material comprises a mesh with two sides that are free to, move, slip, or stretch with respect to each other.

88. The device of any one of claims 1-87, wherein the fibrin or fibrinogen attached to the surface is natural, recombinant, or post-translationally modified.

89. The device of claim 88, wherein the fibrin or fibrinogen is resistant to fibrinolysis/clot breakdown.

90. A method of sealing a wound comprising inserting the device of any one of claims 1-89 into a space of the wound.

91. The method of claim 90, wherein the method further comprises closing the wound.

92. The method of claim 91, wherein the wound is closed by a medical device or tool comprising surgical graspers, hooks, forceps, robot, sutures, staples, tacks, synthetic, biologic glue, or strips of tape.

93. The method of claim 91, wherein the wound is closed by firm pressure.

94. The method of any one of claims 90-93, wherein the method further comprises dispersing an agent.

95. The method of claim 94, wherein the agent comprises fibrinolysis inhibitors, fibrin or fibrinogen modifying agents, plasmin inhibitors, inhibitor of anticoagulants, platelet activating agents, or any combinations thereof.
